(19) United States
(12) Patent Application Publication
(10) Pub. No.: US 2011/0166208 A1
(43) Pub. Date: Jul. 7, 2011

(54) Nucleotide and Amino Acid Sequences for Calmodulin Protein Methyltransferase

(75) Inventors: Robert L. HOUTZ, Lexington, KY (US); Roberta Magnani, Lexington, KY (US); Lynette M.A. Dirk, Lexington, KY (US)

(73) Assignee: University of Kentucky, Lexington, KY (US)

(21) Appl. No.: 12/757,388

(22) Filed: Apr. 9, 2010

Related U.S. Application Data
(60) Provisional application No. 61/167,950, filed on Apr. 9, 2009.

(51) Int. Cl. A61K 48/00; C07H 21/04; C12N 15/63; C12N 1/21; C12N 9/10; C07K 14/435; A61P 25/00; A61P 15/00; C12Q 1/68

(52) U.S. Cl. 514/44 R; 536/23.2; 435/320.1; 435/252.33; 435/193; 530/350; 435/6.11

(57) ABSTRACT

The present invention provides nucleic acid and amino acid sequences for calmodulin protein methyltransferase. The present invention also provides diagnostic tools and methods of using the present invention to diagnose and treat diseases and conditions linked with calmodulin methyltransferase, as well as methylated calmodulin intermediates.

### GOVERNMENT RIGHTS

This work was supported in part by the Department of Energy, DOE contract DE-FG02-92ER20075. The United States government may have certain rights in the invention. Support was also provided by the Kentucky Science and Engineering Foundation, through Grant No. KSEF-1526-RDE-010.

### FIELD OF THE INVENTION

The present invention provides nucleic acid and amino acid sequences for calmodulin protein methyltransferase. The present invention also provides diagnostic tools and methods of using the present invention to diagnose and treat diseases and conditions linked with calmodulin methyltransferase, as well as methylated calmodulin intermediates.

### BACKGROUND

Calmodulin is the most widely distributed and the most common mediator of calcium effects. Many of the proteins that bind with calmodulin are unable to bind calcium themselves, and thus use calmodulin as a calcium sensor and signal transducer. Calmodulin also utilizes calcium stores in the endoplasmic reticulum, sarcoplasmic reticulum, and, in plants, the vacuole.

Calmodulin undergoes a conformational change upon binding to calcium, which enables it to bind to specific proteins for a specific response. Calmodulin can bind up to four calcium ions, and can undergo post-translational modifications, such as phosphorylation, acetylation, methylation and proteolytic cleavage, each of which can potentially modulate its actions (Stevens (1983) Can J Biochem Cell Biol., 61:906-10.).

Calmodulin contains two pairs of EF-hand domains, located in the N and C-terminal halves of the molecule, connected by a flexible central helix. Binding of Ca⁺² to the EF-hand domains of calmodulin induces a conformational change in the protein. In the presence of a target peptide, a further conformational change results in the flexible central helix being partially unwound and wrapped around the target peptide. In this manner, calmodulin interacts with a wide variety of target proteins.

The binding of Ca⁺² to calmodulin induces conformational changes in the protein that permits it to interact with, and regulate the activity of over 100 different proteins. Thus, calmodulin interactions are involved in a multitude of cellular processes including, but not limited to, gene regulation, DNA synthesis, cell cycle progression, mitosis, cytokinesis, cytoskeletal organization, muscle contraction, signal transduction, ion homeostasis, exocytosis, and metabolic regulation. Calmodulin mediates processes such as inflammation, metabolism, apoptosis, muscle contraction, intracellular movement, short-term and long-term memory, nerve growth and the immune response. Calmodulin is a key mediator of calcium-dependent signaling and is subject to regulatory post-translational modifications including trimethylation of Lys 115, a solvent accessible position.

Site-specific methylation of lysyl residues by protein methyltransferases is an important determinant in enzyme activity and protein-protein interactions. Post-translational methylation of protein lysyl residues has emerged as an important determinant of protein-protein interactions. This modification is catalyzed by protein lysine methyltransferases (PKMTs) which, in conjunction with proteins with binding domains that recognize methylated lysyl residues (Taverna et al., Nat Struct Mol Biol, 14:1025-1040 (2007), and enzymes that reverse lysyl methylation (Klose et al., Nat. Rev. Genet, 7:715-727 (2006); Klose et al, Nat Rev Mol Cell Biol, 8:307-318 (2007); Shi et al., Mol Cell, 25:1-14 (2007), have important roles in regulating several cellular and developmental processes. A limited number of studies have demonstrated that the methylation state of calmodulin can change in developmentally and tissue dependent manners (Oh et al., Plant Physiol., 93, 880-887 (1990); Rowe et al., J Biol Chem, 261, 7060-7069 (1986); Takemori et al., Proteomics, 7, 2651-2658 (2007)), influence the activator properties of calmodulin with target enzymes (Roberts et al., Plant Physiol., 75:796-798 (1984)), and cause phenotypic changes in growth and developmental processes at the level of a whole organism (Roberts et al., Proc Natl Acad Sci, 89, 8394-8398 (1992)). These observations suggest that calmodulinmethylation could be a dynamic mechanism attenuating the interaction of calmodulin with target proteins influencing a plethora of eukaryotic cellular and developmental processes. Despite several decades of research providing biochemical characterization and data regarding physiological importance, the particular protein methyltransferase responsible for formation of trimethyllysine-115 in calmodulin has remained unknown in terms of nucleotide and amino acid sequence.

Several articles have proven the existence of a protein that shows a strong methylation activity toward calmodulin, but the nucleotide or amino acid sequence have not been identified. (J Biol Chem. 1996 May 31; 271(22):12737-43; Biochemistry. 1993 Dec. 21; 32(50):13974-80; Biochim Biophys Acta. 1994 Mar. 2; 1199(2):183-94. Dirk et al., (2006) in The Enzymes (Tamanoi, F., and Clarke, S., eds) pp. 179-228, Elsevier Academic Press, Burlington, Mass.). Other than an association with mental retardation in a hypotonia-cystinuria syndrome, the sequence has not been connected to any known protein and its function was undiscovered (Curr Mol Med. 2008 September; 8(6):544-50. J Med Genet. 2008 May; 45(5):314-8. Epub 2008 Jan. 30.)

Due to the important nature of calmodulin, it would be very useful to determine the nucleic acid and amino acid sequences of a protein which shows strong methylation activity towards calmodulin.

### SUMMARY OF THE INVENTION

The present invention provides isolated polynucleotide sequences encoding calmodulin protein methyltransferases comprising the amino acid sequence of SEQ ID NO. 1, 2, 3 and/or homologous sequences thereof. The homologous sequences may have 80% or more homology to SEQ ID NO: 1, 2 and/or 3. The homologous sequences may have 80%, 85%, 90%, 95% or more homology to SEQ ID NO: 1, 2 and/or 3. Also provided is a composition comprising the polynucleotide, as well as an isolated polynucleotide sequence which is complementary to former polynucleotide sequences. Also provided is an expression vector containing the polynucleotide sequences, as well as a host cell containing the expression vector.

In a further embodiment, the present invention provides a method for producing a polypeptide comprising the amino acid sequence of SEQ ID NO: 1, 2, or 3, comprising the steps of culturing the host cell of claim 5 under conditions suitable for the expression of the polypeptide; and recovering the polypeptide from the host cell culture using affinity chromatography with immobilized calmodulin.

In another embodiment, the present invention provides a method of diagnosing a disease or condition relating to the expression of calmodulin protein methyltransferase, comprising utilization of polynucleotide sequences capable of hybridizing to SEQ ID NO: 1, 2, or 3 or utilization of antibodies raised to the amino acid sequences corresponding to SEQ ID NOs: 1, 2, or 3.

In a further embodiment, the present invention provides a method of treating a disease or condition relating to the expression of calmodulin protein methyltransferase, comprising administering a pharmaceutically effective amount of a composition comprising the sequence of SEQ ID NO:1, 2, and 3. The disease or condition may be of the brain and testes. The disease or condition may be related to hypotonia-cystinuria syndrome or mental retardation.

In another embodiment, the present invention provides a method for generating two forms of calmodulin which vary only in the presence or absence of a trimethyllysyl residue at position 115 and its use to determine changes in interaction with other proteins as well as use of such intermediate to evaluate changes in the activity of the interacting proteins.

### BRIEF DESCRIPTION OF THE DRAWINGS

FIG. 1 shows SDS-PAGE separation of a protein fraction enriched in calmodulin MT activity from lamb testis. A candidate polypeptide was photolabeled with [³H-methyl]AdoMet as shown.

FIG. 2 shows putative peptides identified from the sample, as isolated from the SDS-PAGE gel and subjected to trypsin digestion, followed by MS-MS analysis. The most closely homologous sequence was an unidentified human clone.

FIG. 3 shows multiple homologs of the gene identified using standard BLAST techniques.

FIG. 4 shows clones of the calmodulin methyltransferase candidate, including clones from Homo sapiens, Rattus norvegicus and Xenopus laevis (FIG. 4).

FIG. 5 shows a high homology and identity of the three sequences with unknown functions.

FIG. 6 shows the results of phosphorimage analysis, showing that the radiolabel from [³H-methyl]AdoMet was incorporated into calmodulin, in the presence of the recombinant calmodulin methyltransferases obtained from SEQ ID NO: 1, 2, and 3.

FIG. 7 shows in vitro assays of bacterially-expressed calmodulin and calmodulin methyltransferase subjected to SDS-PAGE. The calmodulin was cut from the gel, trypsinolized and the peptides analyzed by MS-MS.

FIG. 8 shows that lysyl residues from hydrolyzed in vitro methylated calmodulin were separated by thin layer chromatography and radiolabeled methyllysyl forms, identified using phosphorimagery.

FIG. 9 shows modeling of calmodulin KMT as a Class I MT and polypeptide sequence alignment. FIG. 9 shows a molecular model of the catalytic core of HsCaM calmodulin PKMT (residues 70 to 292) based on the homology with ribosomal L11 protein lysine methyltransferase (PRMA; 2ZBQ.pdb). The loops highlighted in red denote the putative AdoMet-binding site. This figure was rendered using PyMOL (http://www.pymol.org/); FIG. 9b shows the polypeptide alignment of Hs, Rn, Bt, Xl, Gg, At, Os, and TcCaM PKMT (Protein Accession #: AAH5373-Homo Sapiens (Hs), NP_{—}001127935-Rattus norvegicus.

### DETAILED DESCRIPTION

The present invention provides nucleic acid and amino acid sequences for calmodulin protein methyltransferase. These sequences have utility for research, diagnostic and therapeutic purposes. Thus, the present invention further provides diagnostic tools and methods of using the present invention to diagnose and treat diseases and conditions linked with calmodulin protein methyltransferase.

Calmodulin is known to interact with multiple proteins and to influence their activities. A summary of those interactions can be found for human at http://www.himap.org/ and http://www.hprd.org (112 interactions listed for human calmodulin methyltransferase which is the only isoform listed with post-translational modification of position 115 [previous convention for the numbering was after methionine removal] by trimethylation; two other isoforms for human calmodulin, 2 and 3).

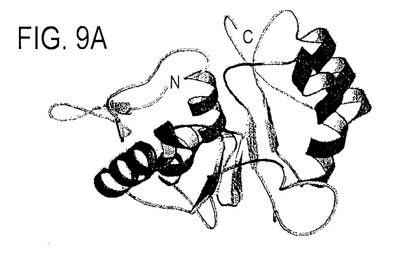

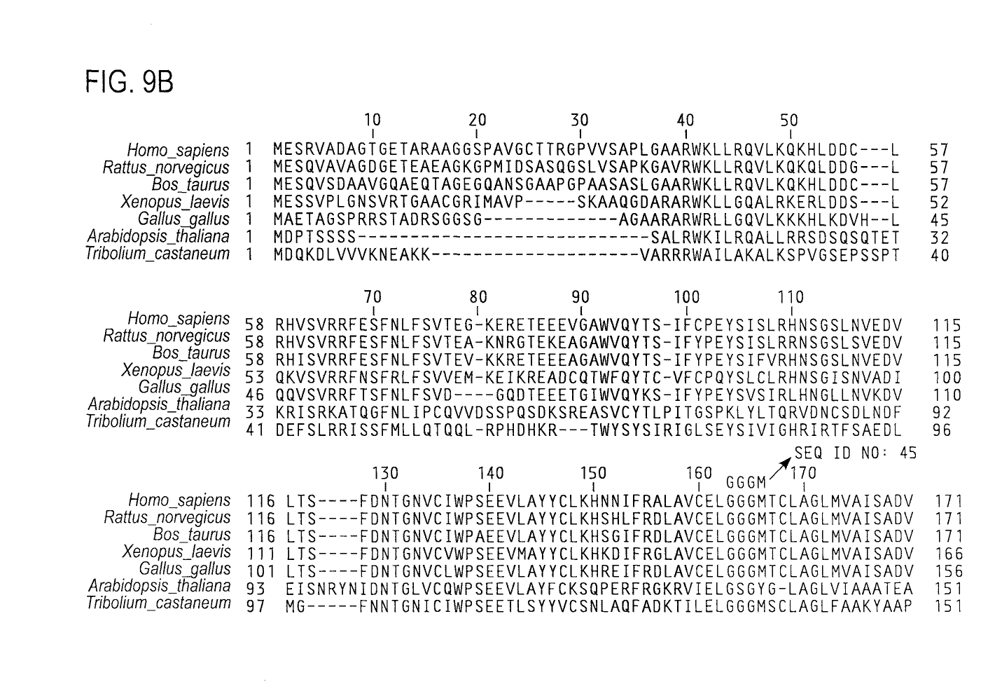

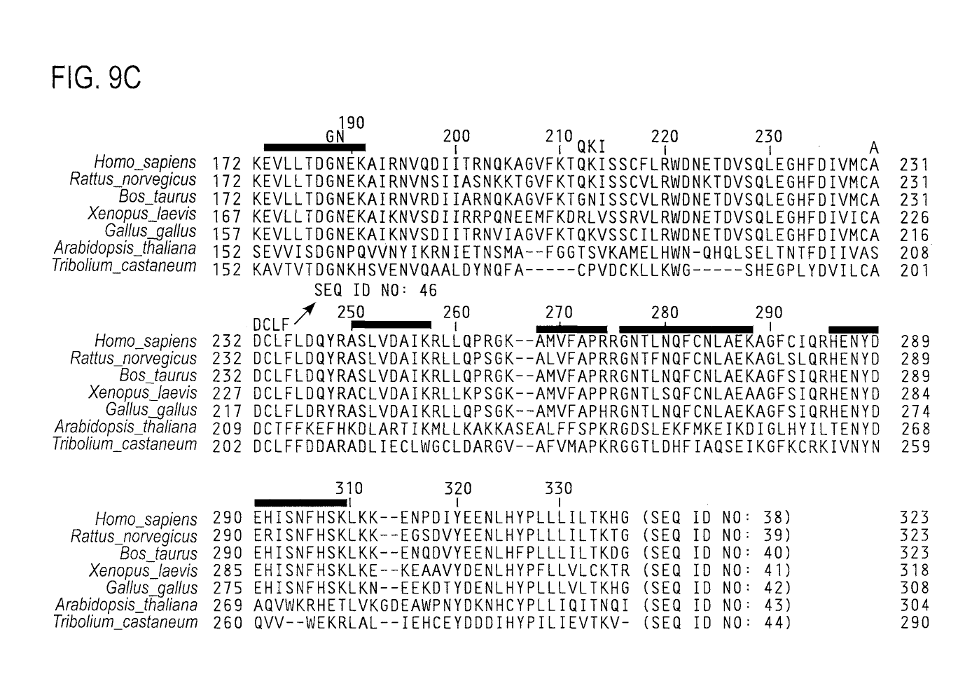

Calmodulin PKMT contains an annotated AdoMet-binding motif found in a large number of Class 1 methyltransferases (Petrossian et al., Mol Cell Proteomics, 8:1516-1526 (2009) including protein arginine methyltransferase (PRMT), protein ribosomal methyltransferase (PRMA), and protein isoaspartyl methyltransferase (PIMT). Using the ESyPred3D server (Lambert et al., Bioinformatics, 18:1250-56 (2002)) and the L11 PRMA as a template (2ZBQ.pdb), a molecular model for calmodulin PKMT corresponding to residues 70 to 292 was generated, corresponding to the catalytic domain of Class I methyltransferases that bind AdoMet (FIG. 9a). The N- and C-terminal regions that flank the catalytic core are predicted to possess ordered secondary structure according to secondary structure prediction programs but display no sequence homology with PRMA, PRMTs, or PIMT. These flanking regions may be responsible for grasping calmodulin and docking Lys-115 into the active site of calmodulin PKMT for trimethylation, as is observed in the ribosomal L11 substrate binding mode of PRMA¹⁵. These regions (Hs N-terminus, A38⁻¹D55, L57⁻N69; C-terminus L299, Y307⁻K321) are also conserved across all calmodulin PKMT orthologues (FIG. 9b) and may demonstrate the highly conserved nature of regions involved in calmodulin binding.

The human gene, c2orf34, that encodes calmodulin KMT is at locus 2p21 that is subject to deletions which are linked to hypotonia-cystinuria syndrome (HCS), an atypical HCS, and the 2p21 deletion syndrome16. The deletions differ in size and the number of genes affected. Patients afflicted by deletion in c2orf34 manifest numerous biochemical symptoms and phenotypes, including a possible deficiency in complex IV of the respiratory chain and mild to moderate mental retardation. Some of the genes involved in the vastly diverse cognitive disorders that go under the name of mental retardation are known to be histone methyltransferases (Kramer et al., Int J Biochem Cell Biol, 41:96-107 (2009)).

There are some annotated discrepancies with Hs calmodulin PKMT, cDNA (Accession BC 053733) has a single predicted amino acid alteration (V209F). However, according to protein alignment, the valine residue is conserved among all mammalian calmodulin PKMTs (FIG. 9b). Though the initially expressed form of Hscalmodulin PKMT was catalytically inactive, alteration of the sequence of the clone to correspond to the gene-predicted valine restored catalytic activity to amounts similar to those observed for other cloned calmodulin KMTs. To date, of the about 2000 reported single nucleotide polymorphisms (SNPs) for this gene, this SNP (rs17855699) has no frequency data established and thus, the significance of this discrepancy remains undetermined, although several other SNPs are apparently associated with specific human genetic backgrounds (http://www.ncbi.nlm.nih.gov/sites/entrez?db=snp). Several gene expression analyses suggest that there could be significant tissue specific differences in c2orf34 expression as well as differences between normal and cancer cell lines (http://www.genecards.org/cgi-bin/carddisp.pl?gene=C2ORF34).

The present invention has found the link between calmodulin protein methyltransferase and its corresponding nucleic acid and amino acid sequences as follows.

SEQ ID NO: 1: Homo sapiens
ATGGAGTCGCGAGTCGCGGACGCTGGGACCGGCGAGACCGCGCGAGCAG
CGGGCGGGAGTCCGGCAGTTGGCTGCACCACTCGGGGGCCCGTAGTCTC
GGCGCCCCTGGGAGCCGCCCGGTGGAAGCTCCTGCGGCAGGTTCTGAAG
CAAAAACACCTGGATGATTGCCTGCGACATGTATCTGTAAGAAGATTTG
AATCATTTAATCTGTTTTCAGTAACAGAAGGCAAAGAAAGGGAAACTGA
AGAGGAGGTTGGTGCATGGGTCCAATATACAAGCATCTTCTGTCCTGAA
TACAGTATCTCCTTAAGGCATAATAGTGGATCCTTGAATGTTGAAGATG
TCCTTACCAGCTTTGACAATACAGGAAATGTTTGCATCTGGCCATCTGA
AGAGGTTTTGGCTTACTACTGCCTCAAGCACAATAATATATTCAGGGCC
CTTGCTGTGTGTGAGCTAGGGGGTGGCATGACATGCTTGGCTGGGCTCA
TGGTTGCTATTTCTGCAGATGTCAAAGAAGTTCTGTTAACTGATGGGAA
TGAAAAGGCCATCAGAAATGTGCAAGACATCATCACAAGGAATCAGAAG
GCTGGTGTGTTTAAGACCCAGAAAATATCAAGCTGCGTTTTACGATGGG
ATAATGAGACAGATGTCTCTCAACTGGAAGGACATTTTGACATTGTTAT
GTGTGCTGACTGCCTGTTTCTGGACCAGTACAGAGCCAGCCTTGTTGAT
GCAATAAAGAGATTACTCCAGCCCAGGGGGAAAGCGATGGTATTTGCCC
CACGCCGAGGGAATACTTTAAACCAGTTTTGCAATCTAGCTGAAAAAGC
TGGTTTCTGTATCCAAAGACATGAAAATTATGATGAACACATTTCAAAC
TTCCACTCCAAGTTGAAAAAGGAAAACCCGGACATATATGAAGAAAACC
TTCATTACCCGCTTCTGCTTATTTTGACCAAACATGGATAG
SEQ ID NO: 2: Rattus norvegicus
ATGGAGTCGCAGGTCGCGGTTGCTGGGGATGGAGAGACTGAGGCAGAGG
CGGGCAAGGGTCCCATGATTGACAGTGCCAGCCAAGGGTCGCTGGTCTC
GGCGCCCAAGGGGGCTGTCCGGTGGAAGCTCCTGCGACAGGTTCTGAAG
CAGAAACAGCTGGATGACGGCCTGAGACATGTATCTGTGAGAAGATTTG
AATCATTTAATCTGTTCTCAGTCACAGAGGCCAAAAACAGGGGAACTGA
AAAAGAGGCTGGTGCGTGGGTCCAGTACACAAGCATCTTTTATCCCGAG
TACAGCATCTCCTTAAGGCGTAATAGTGGATCCTTGAGTGTCGAAGACG
TACTGACCAGCTTTGACAACACAGGGAATGTCTGCATCTGGCCGTCTGA
GGAGGTGTTGGCTTACTACTGCCTCAAGCACAGCCATTTATTCAGGGAC
CTTGCTGTGTGTGAGCTAGGGGGTGGCATGACATGCTTGGCTGGGCTCA
TGGTTGCTATTTCTGCAGATGTCAAAGAAGTTCTGCTAACTGATGGGAA
TGAAAAGGCCATCAGAAATGTGAACAGCATCATCGCAAGCAACAAGAAG
ACTGGCGTGTTCAAGACTCAGAAAATATCAAGCTGCGTTTTACGATGGG
ATAATAAGACAGATGTCTCTCAACTGGAAGGACATTTTGACATTGTTAT
GTGTGCTGACTGCCTGTTCCTGGACCAGTACAGAGCCAGCCTTGTTGAT
GCAATAAAGAGATTACTCCAGCCCTCAGGGAAAGCATTGGTATTTGCCC
CACGCCGAGGGAATACTTTCAACCAGTTTTGCAATCTAGCAGAAAAAGC
CGGTCTCTCCCTCCAAAGACATGAAAATTACGATGAGCGCATTTCAAAC
TTCCACTCCAAGTTAAAAAAAGAAGGGTCGGACGTATATGAAGAAAATC
TCCACTACCCACTTCTACTTATTTTAACCAAAACGGGATAG
SEQ ID NO: 3: Xenopus laevis
ATGGAGAGCTCGGTGCCGCTTGGGAATAGTGTCAGGACGGGAGCAGCGT
GTGGAAGGATAATGGCTGTTCCCAGTAAAGCTGCGCAAGGAGACGCCAG
AGCCCGCTGGAAGCTATTGGGACAGGCCCTTAGAAAAGAGCGCTTGGAC
GACAGTCTCCAGAAAGTGTCTGTTCGAAGATTCAATTCCTTTCGTCTGT
TCTCAGTGGTGGAAATGAAAGAGATAAAACGAGAAGCCGATTGCCAAAC
CTGGTTTCAGTACACCTGTGTGTTCTGCCCTCAGTACAGCCTGTGCTTA
AGACATAATTCTGGCATCTCCAACGTGGCCGACATCCTCACAAGTTTCG
ACAACACCGGAAATGTCTGTGTGTGGCCTTCTGAAGAGGTAATGGCCTA
CTACTGCCTCAAGCATAAAGATATATTCAGGGGCCTTGCTGTGTGTGAA
CTAGGGGGTGGCATGACATGCTTGGCTGGGCTCATGGTCGCAATTTCTG
CCGATGTCAAGGAAGTCCTATTGACGGACGGAAACGAAAAGGCCATCAA
AAATGTTTCTGATATAATAAGAAGACCCCAAAATGAAGAAATGTTTAAA
GATCGGCTTGTTTCGAGCAGAGTTCTGCGATGGGATAATGAGACAGATG
TCTCTCAACTGGAAGGACATTTTGACATTGTTATTTGCGCAGACTGCTT
GTTTCTGGACCAGTACAGAGCCTGCCTTGTTGATGCAATAAAGAGATTA
CTGAAGCCCAGTGGAAAAGCGATGGTATTTGCTCCACCGAGGGGGAATA
CTTTAAGCCAGTTTTGCAATCTAGCGGAGGCGGCTGGTTTTTCCATCCA
AAGACATGAAAATTACGATGAGCACATTTCCAACTTCCACTCCAAGCTG
AAAGAGAAAGAAGCCGCAGTCTACGACGAAAACCTCCACTATCCCTTTC
TACTGGTTCTTTGTAAAACAAGGTAG

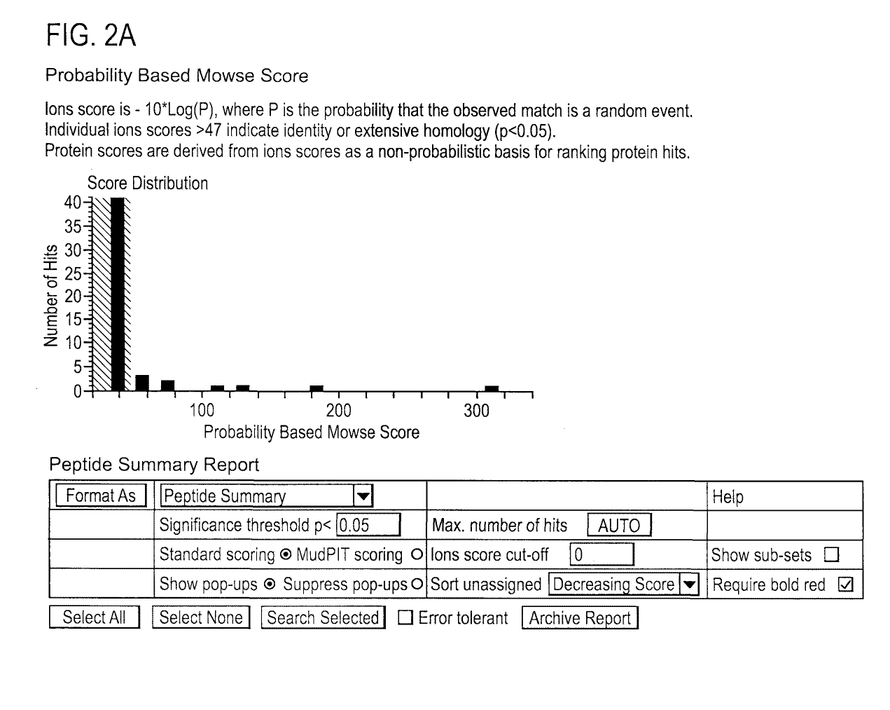

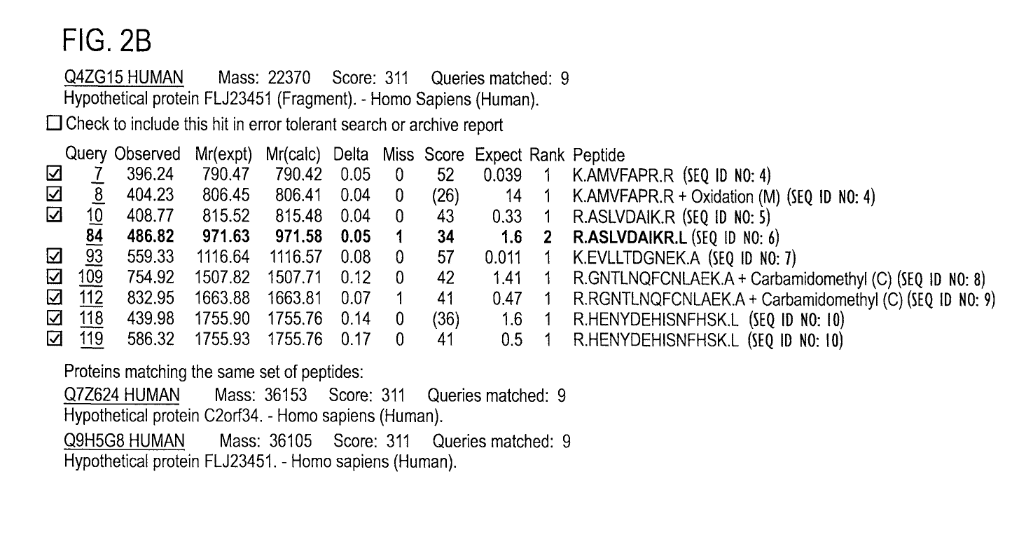

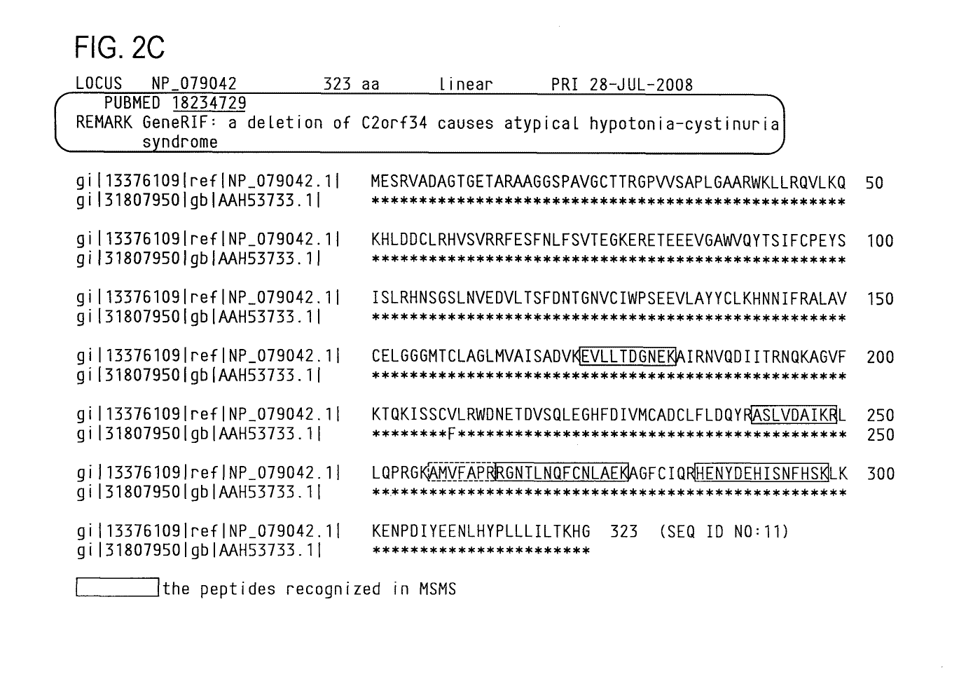

Following analysis of a protein fraction from lamb testis enriched in calmodulin methyltranferase activity, and protein separation, a candidate protein was isolated and analyzed (see Example 1). Tryptic polypeptides were isolated and identified and are summarized in FIG. 2. A Mascot search (Mascot Daemon; Matrix Science) using the probability based Mowse score provided a peptide summary report that revealed the closest homology as a human clone of a protein with unknown function. (“Probability Based Mowse Score: Ions score is −10*Log(P), where P is the probability that the observed match is a random event. Individual ions scores >47 indicate identity or extensive homology (p<0.05). Protein scores are derived from ions scores as a non-probabilistic basis for ranking protein hits.”)

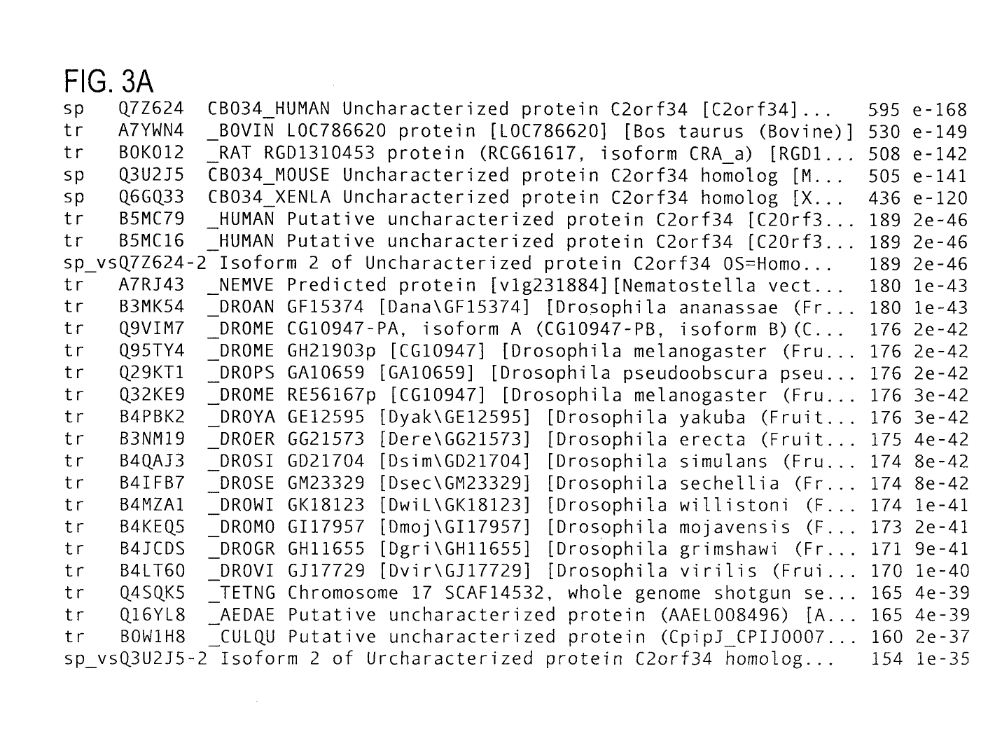

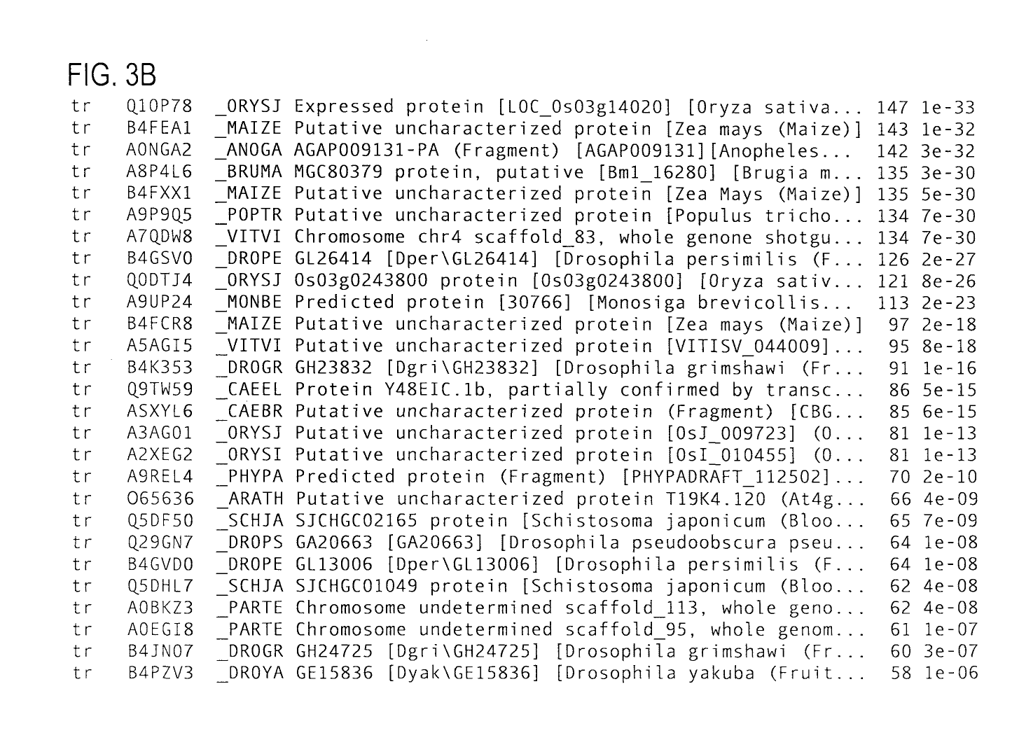

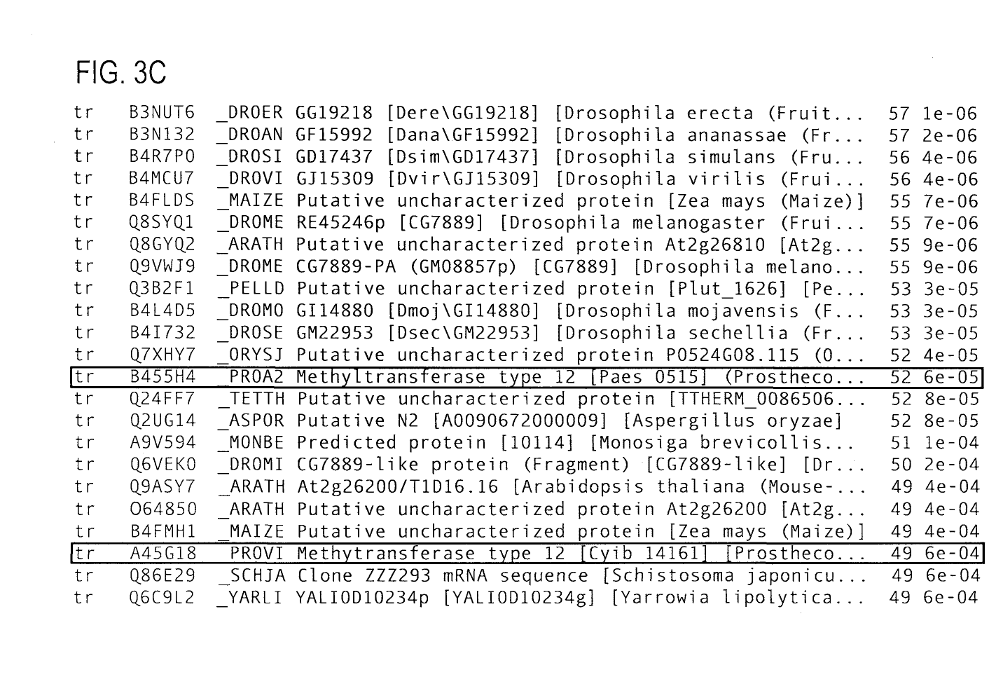

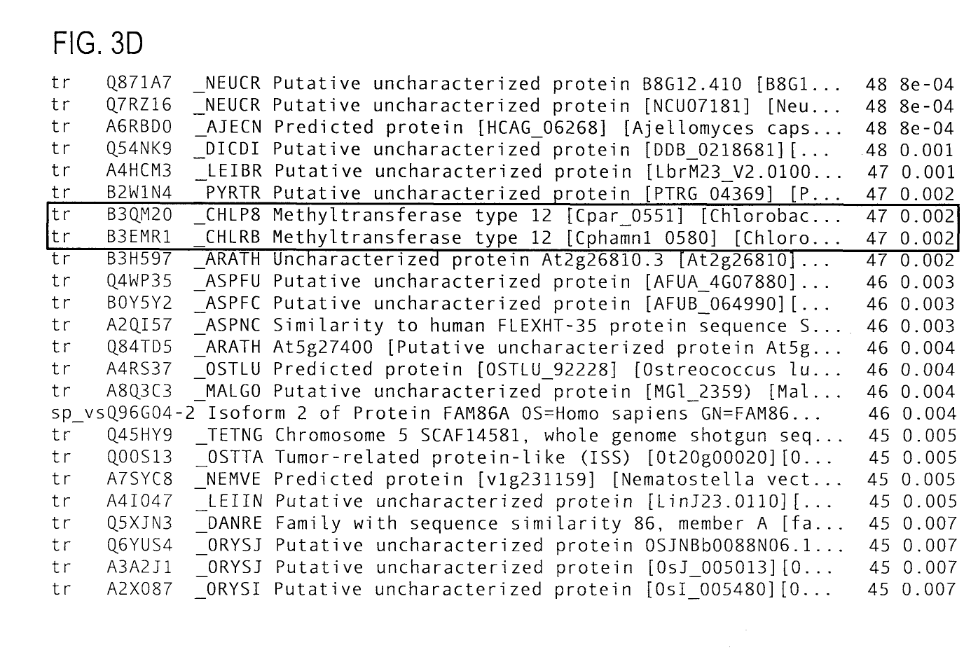

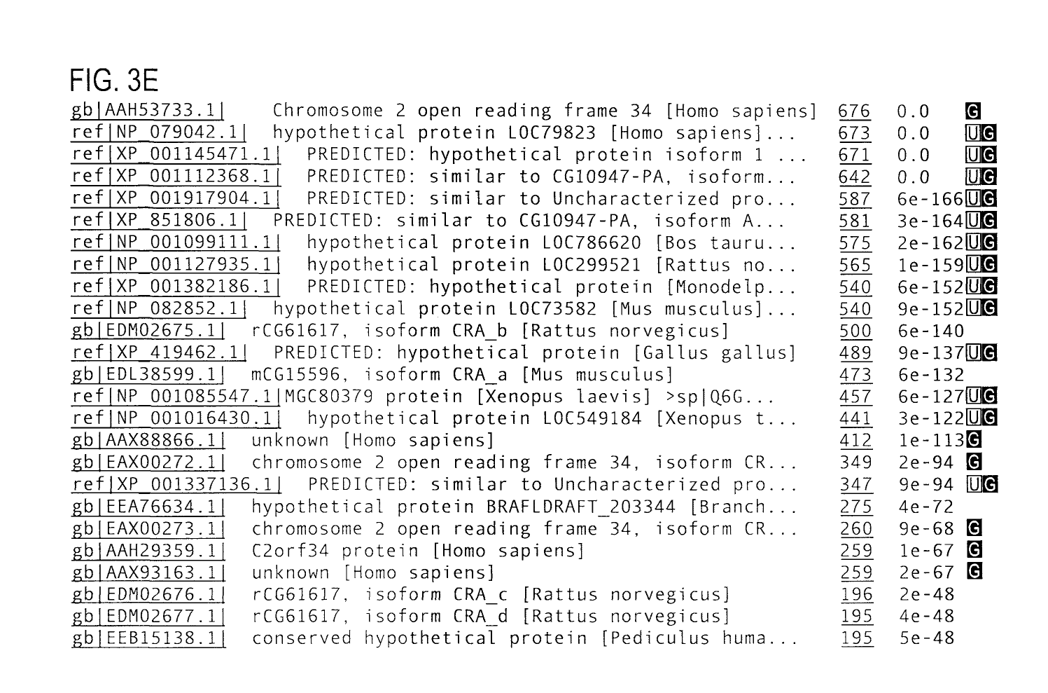

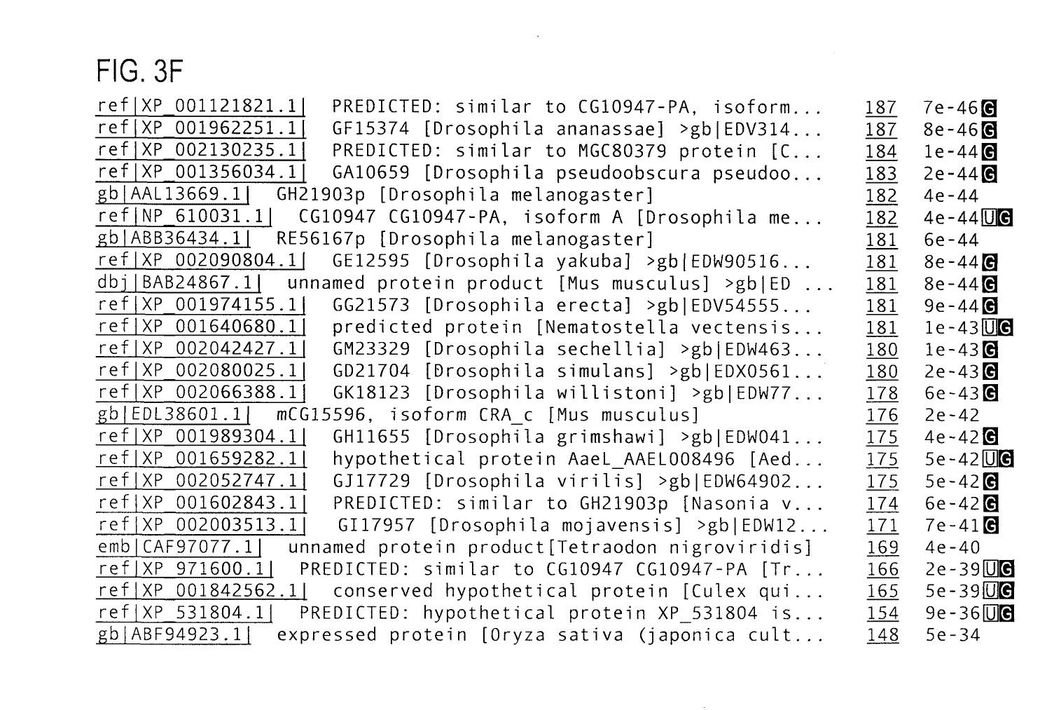

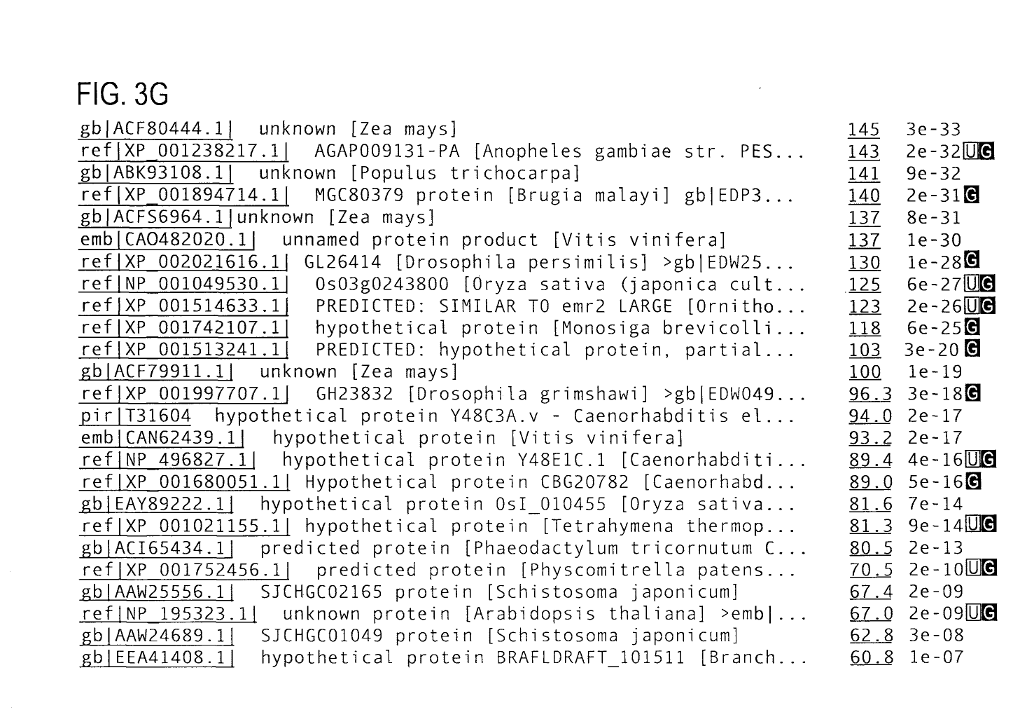

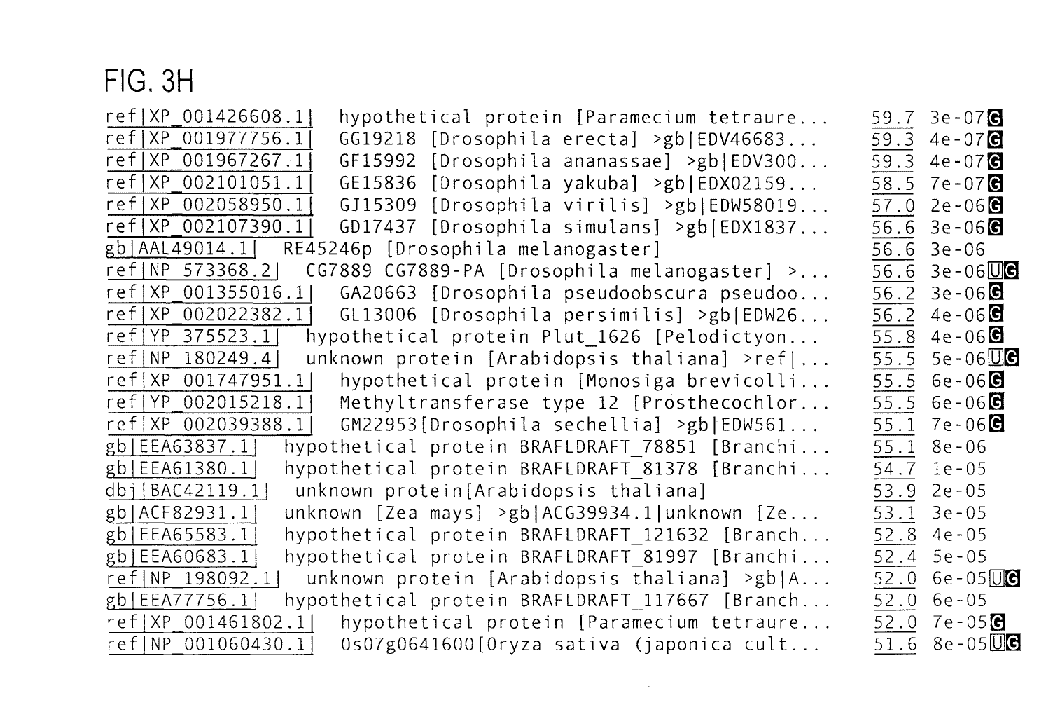

Although protein function was unknown at this point, an AdoMet binding site had been electronically assigned. Multiple homologs of the gene from many species were identified using standard BLAST techniques. The proteins identified in a standard BLAST search are shown in FIG. 3.

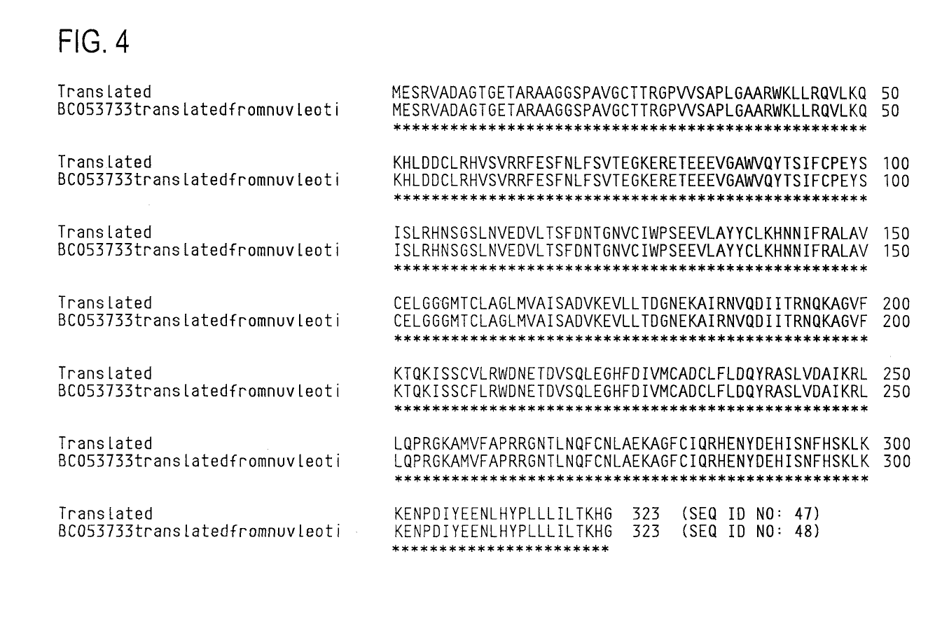

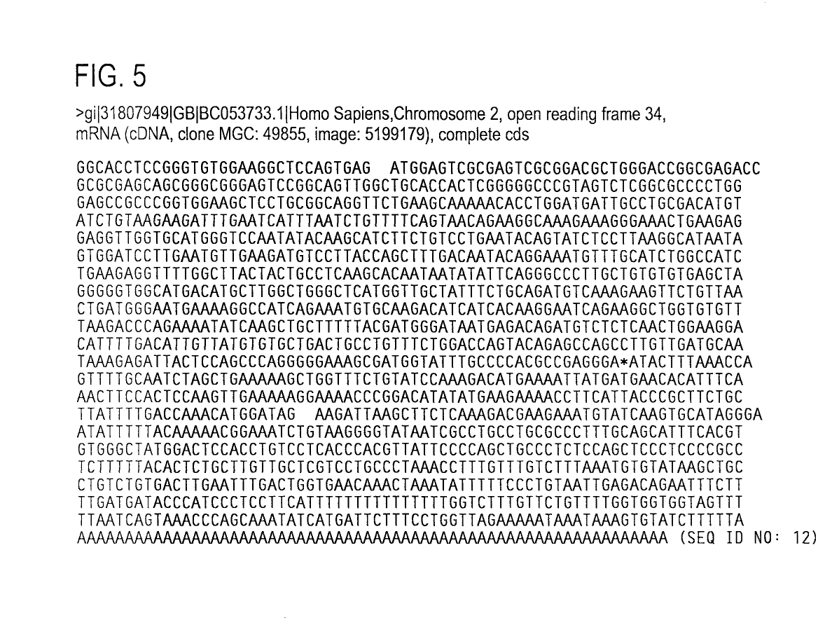

Clones of the calmodulin methyltransferase candidates were analyzed. Clones analyzed included clones from Homo sapiens, Rattus norvegicus and Xenopus laevis as shown in FIG. 4. FIG. 5 provides sequence alignments showing that the three sequences show high homology and identity, all with unknown function.

After subcloning into bacterial expression vectors, all three proteins were produced in E. coli and purified using affinity chromatography (Han, C. H., Richardson, J., Oh, S. H., and Roberts, D. M. (1993) Biochemistry 32, 13974-13980). Each of the proteins exhibited robust methyltransferase activity (in vitro) toward non-methylated forms of calmodulin (those produced by bacteria which do not contain calmodulin methyltransferase).

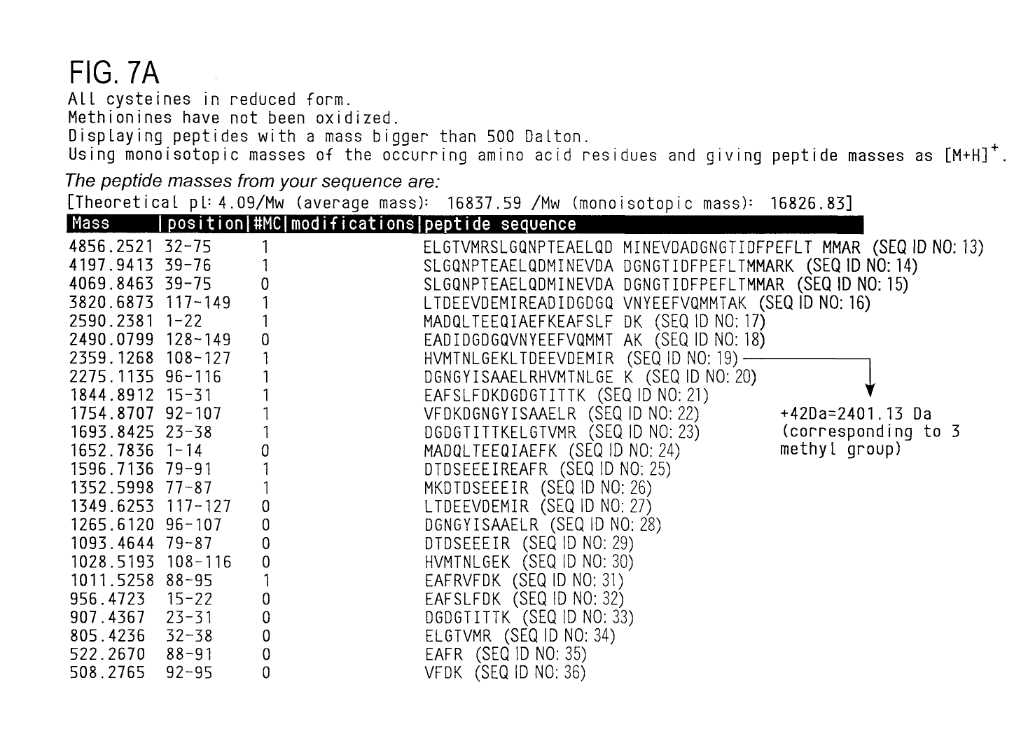

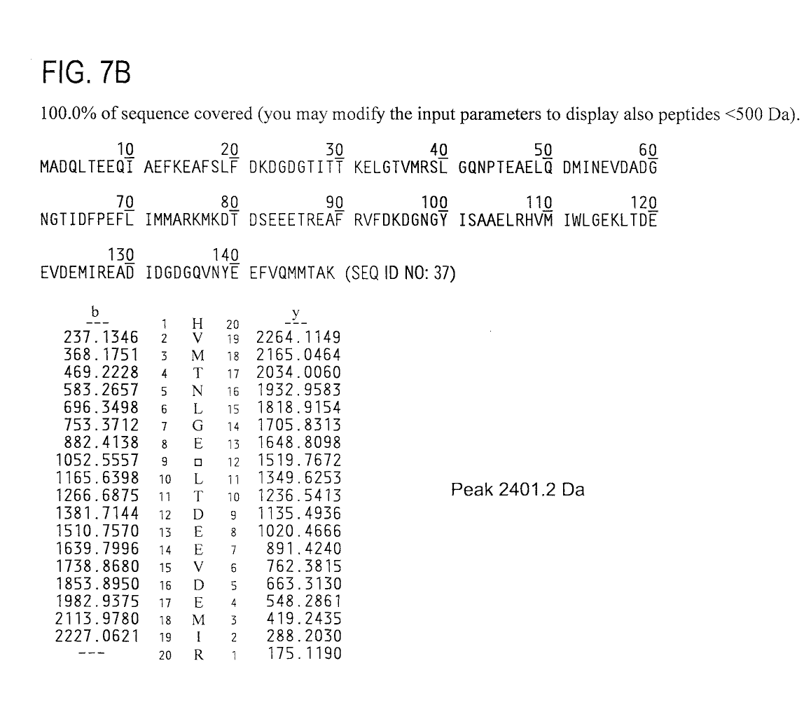

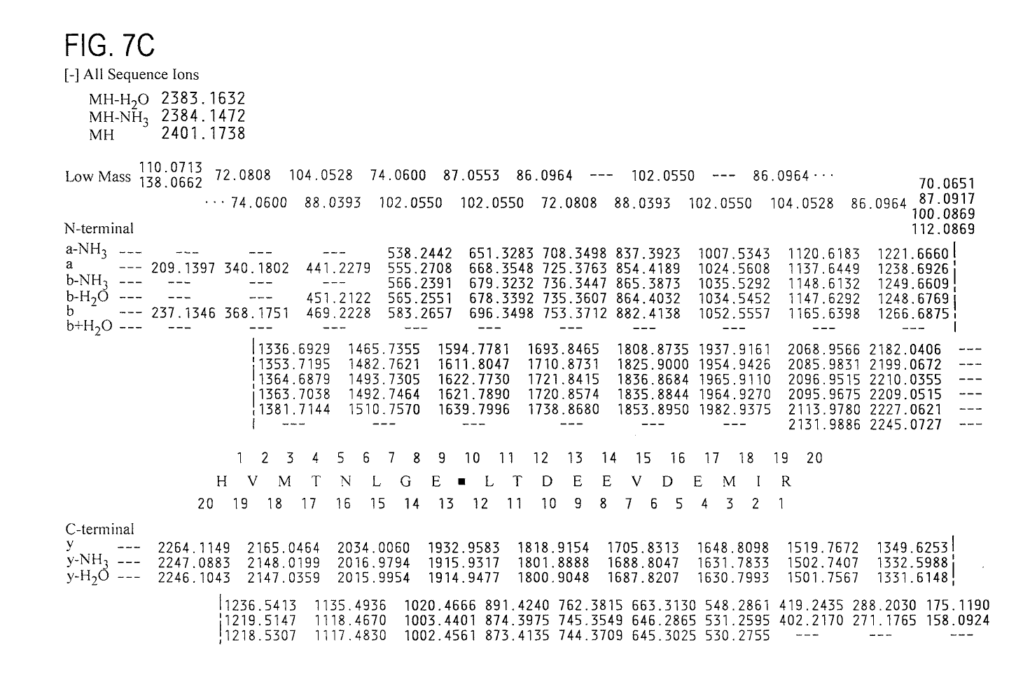

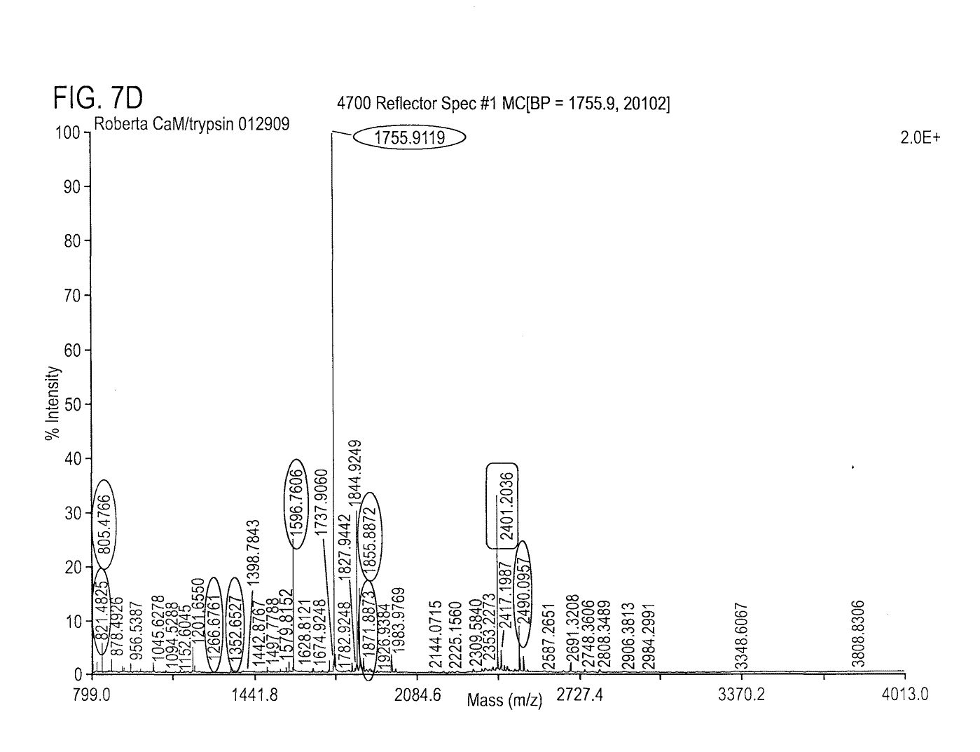

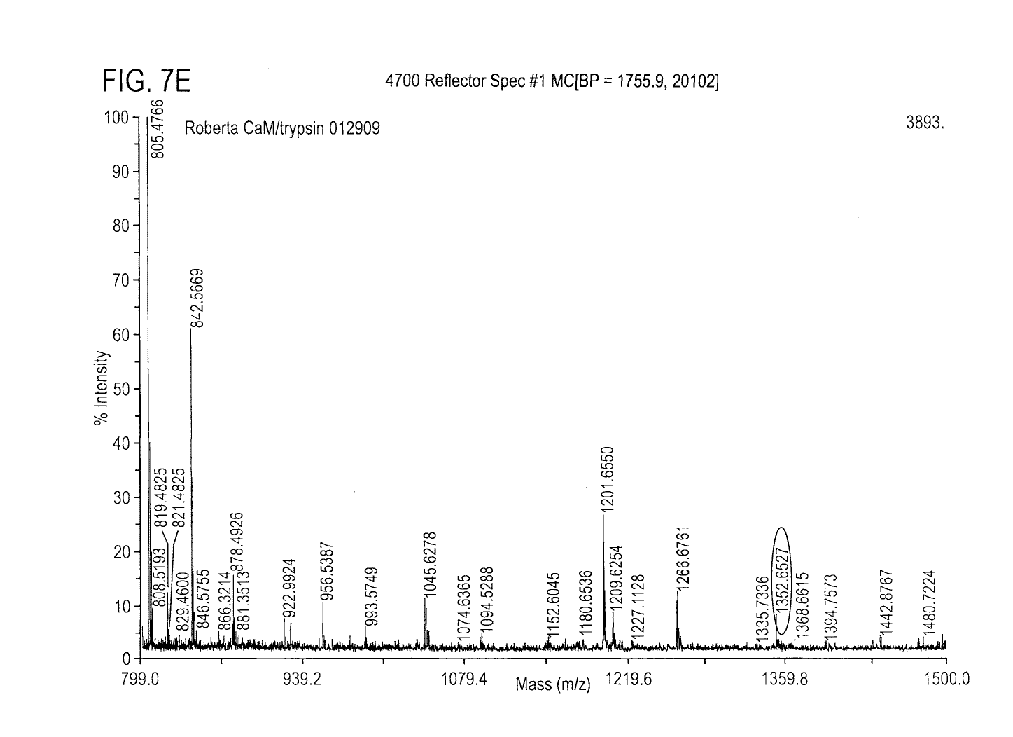

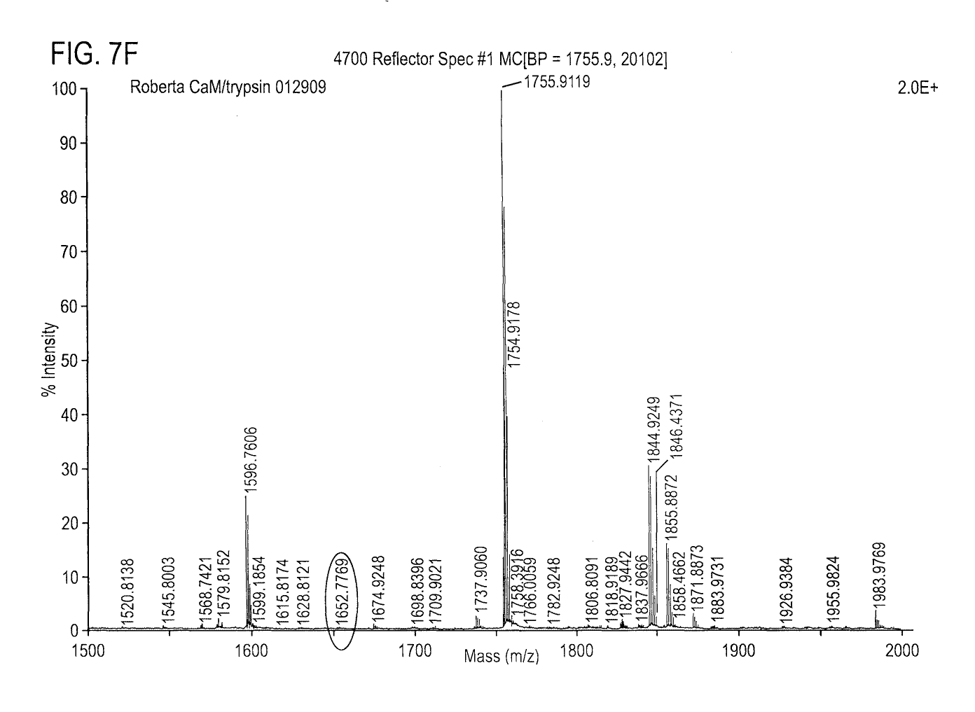

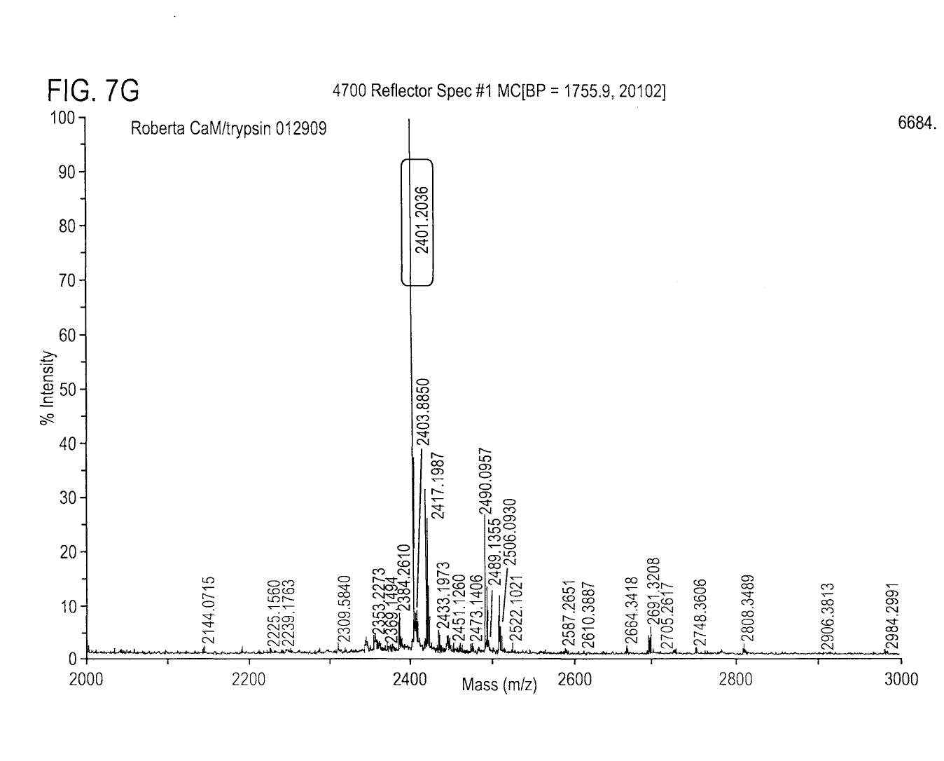

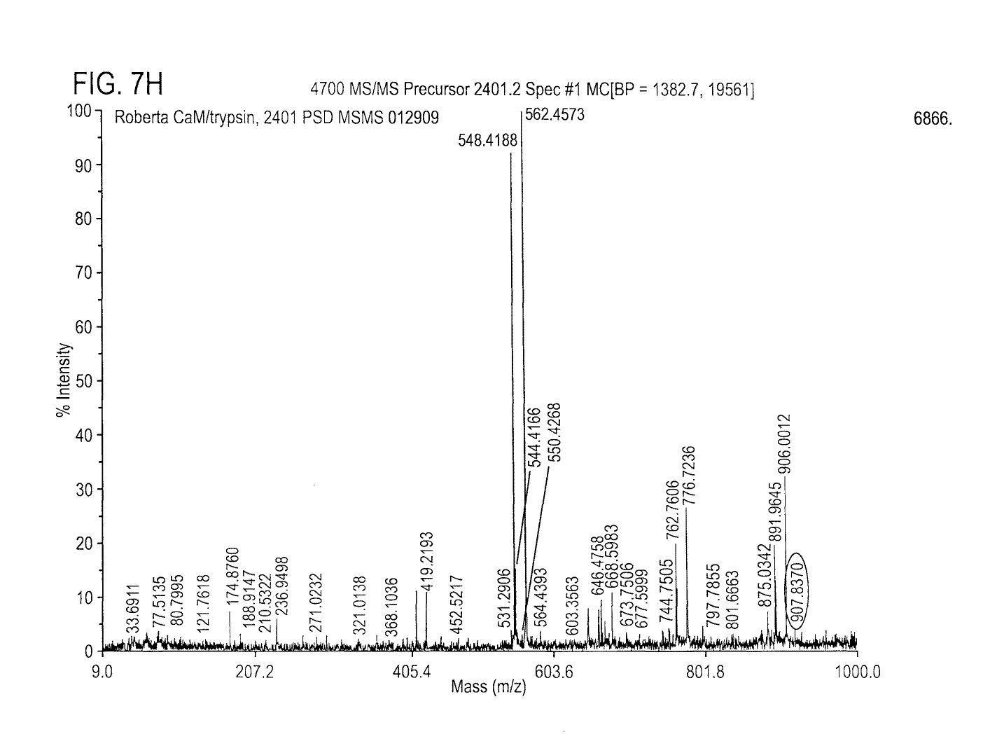

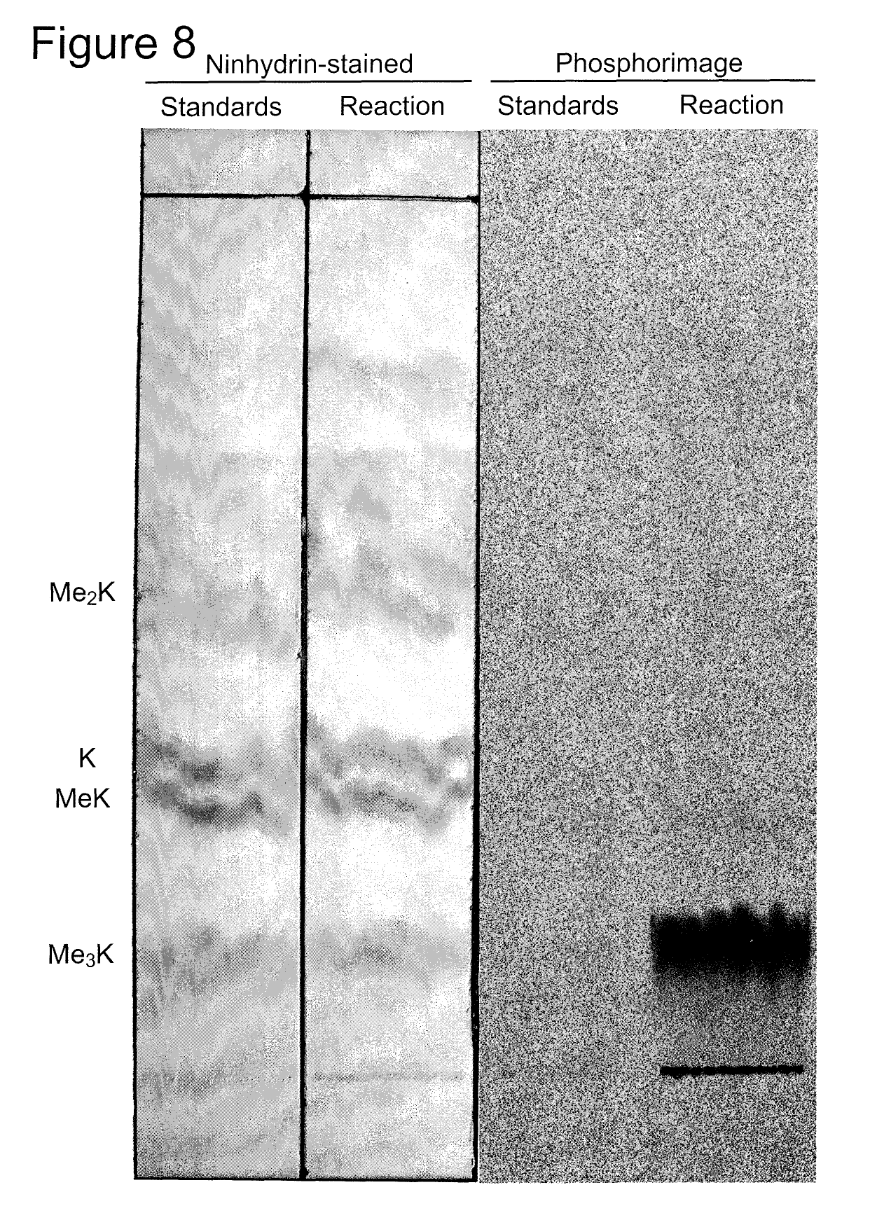

To confirm the site of the methylation in calmodulin, in vitro assays of bacterially-expressed calmodulin and calmodulin methyltransferase were analyzed. The peptides analyzed by MS-MS are as shown in FIG. 7. The spectra included a peak of 2401 Da, corresponding to the peptide containing Lys115 modified with three additional methyl groups. A peptide with a mass of 1028 Da, representative of the same peptide without methylation, was not present in the spectra. Only Lys 115 was methylated. Trimethylation of Lys-115 in calmodulin was also documented by complete hydrolysis of methylated calmodulin and analyses of methylated lysyl residues. Only trimethyllysine was found (FIG. 8).

The molecular structure of calmodulin methyltransferases may be used for the design of pharmacologically active compounds, for the treatment of diseases and conditions linked to calmodulin methyltransferase expression. The sequences of the present invention may also be useful in medically-related gene therapies.

The present invention further provides diagnostic tools for detecting the calmodulin methyltransferase protein. Molecular probes and methods for detecting calmodulin methyltransferase protein, DNA, and RNA may be used in the identification and diagnosis of mutations in the associated gene in humans. Methods of preparing such probes and constructs using the sequences provided herein are well known to those of skill in the art. Antibodies to calmodulin methyltransferase may also be useful for the detection or lack thereof of calmodulin methyltransferase. Methods of preparing such antibodies and constructs using the sequences provided herein are also well known to those of skill in the art.

Calmodulin interacts with and influences the activity of hundreds of proteins in the cell. Calmodulin methylation status which is determined by calmodulin methyltransferase activity regulates some of these interactions. Accordingly, any disease or condition which is caused or is affected by calmodulin methyltransferase activity or lack thereof, or by calmodulin methylation status, could be diagnosed using the tools of the present invention.

For example, one clinical disease which has been linked to a lack of calmodulin methyltransferase expression is mental retardation in association with hypotonia-cystinuria syndrome (Chabrol, B., Martens, K., Meulemans, S., Cano, A., Jaeken, J., Matthijs, G., and Creemers, J. W. (2008) J Med. Genet. 45, 314-318; Martens, K., Jaeken, J., Matthijs, G., and Creemers, J. W. (2008) Curr. Mol. Med. 8, 544-550; Parvari, R., Gonen, Y., Alshafee, I., Buriakovsky, S., Regev, K., and Hershkovitz, E. (2005) Genomics 86, 195-211).

Hypotonia-cystinuria syndrome is genetic disorder characterized by reduced muscle tone, growth hormone deficiency and unusual facial appearance. It is also characterized by mental retardation when deletions/mutations are present in the flanking gene which codes for calmodulin methyltransferase. Failure to thrive occurs during the first years of life but is replaced by rapid weight gain in later childhood. Hypotonia-cystinuria syndrome results from deletions in chromosome 2p21. The sequences and methods of the present invention may be used to provide tools for the diagnosis of atypical hypotonia-cystinuria syndrome and the related mental retardation.

In addition, given the relative high levels of expression of calmodulin methyltransferases, as well as calmodulin itself, in brain and testes, diseases associated with brain and testes may show altered calmodulin methyltransferase activity.

The present invention further provides compositions for and methods of treatment for diseases and conditions associated with the activity of calmodulin methyltransferase.

The present invention also provides an intermediate form of calmodulin. Native forms of calmodulin have two sites of phosphorylation and one methylation site. With the calmodulin methyltransferase sequence, a coexpression E. coli host is made. The methylated-only form of calmodulin would be very useful in research, and cost-effective, since it provides for the only known mechanism to create methylated versus non-methylated versions of calmodulin without other post-translational modifications.

The sequences of the present invention may also be used for diagnosis and treatment in plants. Manipulation of the methylation status of calmodulin in plant results in an increase in the hypersensitive disease resistance response to pathogen invasion. Thus, gene knockouts or gene silencing may be useful in creating disease resistant plants.

The present invention provides a method for producing a polypeptide comprising the amino acid sequence of SEQ ID NO: 1, 2, or 3. The method comprises the steps of culturing a host cell under conditions suitable for the expression of the polypeptide; and recovering the polypeptide from the host cell culture using affinity chromatography with immobilized calmodulin (see the protocols of Klee, C. B. and Krinks, M. H. (1978) Biochemistry 17, 120-126; and Han, C. H., Richardson, J., Oh, S. H., and Roberts, D. M. (1993) Biochemistry 32, 13974-13980).

### Example 1

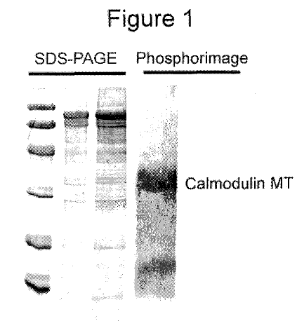

A protein fraction enriched in calmodulin MT activity from lamb testis was isolated using the procedure described in (Han, C. H., Richardson, J., Oh, S. H., and Roberts, D. M. (1993) Biochemistry 32, 13974-13980) Among the proteins separated by SDS-PAGE, a candidate was photolabeled with [³H-methyl]AdoMet (FIG. 1). The candidate protein was isolated from the gel and subjected to trypsin digestion followed by MS-MS analysis. Putative peptides thus identified are summarized in FIG. 2 and the most closely homologous sequence was a human clone (FIG. 2). The protein function was unknown, though an AdoMet binding site had been electronically assigned. Multiple homologues of the gene could be identified using standard BLAST techniques (FIG. 3). cDNA clones from Homo sapiens (Hs), Rattus norvegicus (Rn), Xenopus laevis (Xi), Tribolium casteaneum, and Arabidopsis thaliana (At) were obtained from Open Biosystems, and after subcloning the coding regions into bacterial expression vectors, the proteins were produced in E. Coli and the enzymes purified using bacterially-expressed calmodulin-Sepharose affinity chromatography (FIG. 4). The three sequences showed a rather high homology and identity all with unknown function (FIG. 5). Each of the proteins exhibited robust methyltransferase activity (in vitro) toward non-methylated forms of calmodulin, (i.e. bacterial produced).

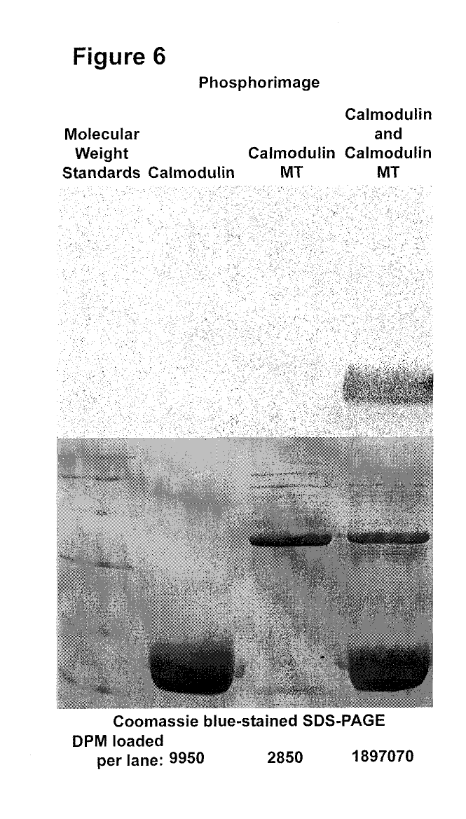

The specific activity of the rat enzyme was 56 nmoles min⁻¹ mg protein⁻¹, which is comparable to the results published for calmodulin MT purified to homogeneity from sheep brain (Han, C. H., Richardson, J., Oh, S. H., and Roberts, D. M. (1993) Biochemistry 32, 13974-13980). Phosphorimage analysis demonstrated that radiolabel from [³H-methyl]AdoMet was incorporated into calmodulin and depended on the presence of the recombinant calmodulin methyltransferases (FIG. 6).

To confirm the site of the methylation in calmodulin, in vitro assays of bacterially-expressed calmodulin and calmodulin methyltransferase were subjected to SDS-PAGE. The calmodulin was cut from the gel, trypsinolized and the peptides analyzed by MS-MS (FIG. 7). The spectra included a peak of 2401 Da, corresponding to the peptide containing Lys115 modified with three additional methyl groups. A peptide with a mass of 1028 Da, representative of the same tryptic peptide without methylation, was not present in the spectra. All peptides containing lysines were identified after digestion with trypsin and Asp-N, demonstrating that only Lys115 was methylated. Trimethylation of Lys-115 in calmodulin was also documented by product analysis. Lysyl residues from hydrolyzed in vitro methylated calmodulin were separated by thin layer chromatography and radiolabeled methyllysyl forms identified using phosphorimagery (FIG. 8).

Specifically, site-specificity was verified using in vitro assays followed by SDS-PAGE and digestion of calmodulin with trypsin and Asp-N protease and identification of peptides by MS/MS. The spectra included a peak at 2401 Da, corresponding to the peptide containing Lys-115 modified by the addition of three methyl groups and thus resistant to tryptic digestion. A peptide with a mass of 1028 Da, representative of the same peptide without methylation, was not present in the spectra. All available lysyl residues were accounted for in the calmodulin peptides obtained from the 2 digestions, demonstrating that only Lys-115 was methylated. These results demonstrate that the nucleotide and associated polypeptide sequences identified here encode protein lysyl methyltransferases specific for Lys-115 of calmodulin (calmodulin PKIVIT; EC 2.1.1.60). Thus, calmodulin protein methyltransferases activity has been linked to specific amino acid and nucleotide sequences.

All references cited in this disclosure are incorporated by reference to the same extent as if each reference had been incorporated by reference in its entirety individually.

While the invention has been described in detail and with reference to specific embodiments thereof, it will be apparent to one skilled in the art that various variations and modifications can be made therein without departing from the spirit and scope thereof. All such variations and modifications are intended to be included within the scope of this disclosure and the present invention and protected by the following claims.

What is claimed is:

1. Isolated polynucleotide sequences encoding calmodulin protein methyltransferases comprising the nucleotide and corresponding amino acid sequences selected from the group consisting of SEQ ID NO: 1, 2, and 3, and homologous sequences thereof.

2. A composition comprising the polynucleotide sequences of claim 1.

3. An isolated polynucleotide sequence which is complementary to the polynucleotide sequences of claim 1.

4. An expression vector containing the polynucleotide sequences of claim 1.

5. A host cell containing the expression vector of claim 4.

6. A method for producing a polypeptide comprising an amino acid sequence selected from the group consisting of SEQ ID NO: 1, 2, 3, and homologous sequences thereof comprising the steps of:
a) culturing the host cell of claim 5 under conditions suitable for the expression of the polypeptide; and
b) recovering the polypeptide from the host cell culture.

7. A method of diagnosing a disease or condition relating to the expression of calmodulin protein methyltransferase, comprising: hybridization of polynucleotide sequences to DNA and RNA sequences selected from the group consisting of SEQ ID NO: 1, 2, 3, and homologous sequences thereof, and antibodies raised against amino acid sequences selected from the group consisting of SEQ ID NO: 1, 2, 3, and homologous sequences thereof.

8. A method of treating a disease or condition relating to the expression of calmodulin protein methyltransferase, comprising administering a pharmaceutically effective amount of a composition of claim 2.

9. The method of claim 8, wherein the disease or condition is a disease or condition of the brain or testes.

10. The method of claim 8, wherein the disease or condition is atypical hypotonia-cystinuria syndrome.

11. A calmodulin intermediate, comprising a methylated form of calmodulin without other post-translational modifications.


SEQUENCE LISTING (48 sequences)

SEQ ID NO: 1 - DNA, 972 bp, Homo sapiens
atggagtcgcgagtcgcggacgctgggaccggcgagaccgcgcgagcagcgggcgggagtccggcagttggctgcaccactcgggggcccgtagtctcggcgcccctgggagccgcccggtggaagctcctgcggcaggttctgaagcaaaaacacctggatgattgcctgcgacatgtatctgtaagaagatttgaatcatttaatctgttttcagtaacagaaggcaaagaaagggaaactgaagaggaggttggtgcatgggtccaatatacaagcatcttctgtcctgaatacagtatctccttaaggcataatagtggatccttgaatgttgaagatgtccttaccagctttgacaatacaggaaatgtttgcatctggccatctgaagaggttttggcttactactgcctcaagcacaataatatattcagggcccttgctgtgtgtgagctagggggtggcatgacatgcttggctgggctcatggttgctatttctgcagatgtcaaagaagttctgttaactgatgggaatgaaaaggccatcagaaatgtgcaagacatcatcacaaggaatcagaaggctggtgtgtttaagacccagaaaatatcaagctgcgttttacgatgggataatgagacagatgtctctcaactggaaggacattttgacattgttatgtgtgctgactgcctgtttctggaccagtacagagccagccttgttgatgcaataaagagattactccagcccagggggaaagcgatggtatttgccccacgccgagggaatactttaaaccagttttgcaatctagctgaaaaagctggtttctgtatccaaagacatgaaaattatgatgaacacatttcaaacttccactccaagttgaaaaaggaaaacccggacatatatgaagaaaaccttcattacccgcttctgcttattttgaccaaacatggatag

SEQ ID NO: 2 - DNA, 972 bp, Rattus norvegicus
atggagtcgcaggtcgcggttgctggggatggagagactgaggcagaggcgggcaagggtcccatgattgacagtgccagccaagggtcgctggtctcggcgcccaagggggctgtccggtggaagctcctgcgacaggttctgaagcagaaacagctggatgacggcctgagacatgtatctgtgagaagatttgaatcatttaatctgttctcagtcacagaggccaaaaacaggggaactgaaaaagaggctggtgcgtgggtccagtacacaagcatcttttatcccgagtacagcatctccttaaggcgtaatagtggatccttgagtgtcgaagacgtactgaccagctttgacaacacagggaatgtctgcatctggccgtctgaggaggtgttggcttactactgcctcaagcacagccatttattcagggaccttgctgtgtgtgagctagggggtggcatgacatgcttggctgggctcatggttgctatttctgcagatgtcaaagaagttctgctaactgatgggaatgaaaaggccatcagaaatgtgaacagcatcatcgcaagcaacaagaagactggcgtgttcaagactcagaaaatatcaagctgcgttttacgatgggataataagacagatgtctctcaactggaaggacattttgacattgttatgtgtgctgactgcctgttcctggaccagtacagagccagccttgttgatgcaataaagagattactccagccctcagggaaagcattggtatttgccccacgccgagggaatactttcaaccagttttgcaatctagcagaaaaagccggtctctccctccaaagacatgaaaattacgatgagcgcatttcaaacttccactccaagttaaaaaaagaagggtcggacgtatatgaagaaaatctccactacccacttctacttattttaaccaaaacgggatag

SEQ ID NO: 3 - DNA, 957 bp, Xenopus laevis
atggagagctcggtgccgcttgggaatagtgtcaggacgggagcagcgtgtggaaggataatggctgttcccagtaaagctgcgcaaggagacgccagagcccgctggaagctattgggacaggcccttagaaaagagcgcttggacgacagtctccagaaagtgtctgttcgaagattcaattcctttcgtctgttctcagtggtggaaatgaaagagataaaacgagaagccgattgccaaacctggtttcagtacacctgtgtgttctgccctcagtacagcctgtgcttaagacataattctggcatctccaacgtggccgacatcctcacaagtttcgacaacaccggaaatgtctgtgtgtggccttctgaagaggtaatggcctactactgcctcaagcataaagatatattcaggggccttgctgtgtgtgaactagggggtggcatgacatgcttggctgggctcatggtcgcaatttctgccgatgtcaaggaagtcctattgacggacggaaacgaaaaggccatcaaaaatgtttctgatataataagaagaccccaaaatgaagaaatgtttaaagatcggcttgtttcgagcagagttctgcgatgggataatgagacagatgtctctcaactggaaggacattttgacattgttatttgcgcagactgcttgtttctggaccagtacagagcctgccttgttgatgcaataaagagattactgaagcccagtggaaaagcgatggtatttgctccaccgagggggaatactttaagccagttttgcaatctagcggaggcggctggtttttccatccaaagacatgaaaattacgatgagcacatttccaacttccactccaagctgaaagagaaagaagccgcagtctacgacgaaaacctccactatccctttctactggttctttgtaaaacaaggtag

SEQ ID NO: 4 - protein, 9 aa, Homo sapiens
KAMVFAPRR

SEQ ID NO: 5 - protein, 10 aa, Homo sapiens
RASLVDAIKR

SEQ ID NO: 6 - protein, 11 aa, Homo sapiens
RASLVDAIKRL

SEQ ID NO: 7 - protein, 12 aa, Homo sapiens
KEVLLTDGNEKA

SEQ ID NO: 8 - protein, 15 aa, Homo sapiens
RGNTLNQFCNLAEKA

SEQ ID NO: 9 - protein, 16 aa, Homo sapiens
RRGNTLNQFCNLAEKA

SEQ ID NO: 10 - protein, 16 aa, Homo sapiens
RHENYDEHISNFHSKL

SEQ ID NO: 11 - protein, 323 aa, Homo sapiens [sequence omitted]

SEQ ID NO: 12 - DNA, 1532 bp, Homo sapiens
ggcacctccgggtgtggaaggctccagtgagatggagtcgcgagtcgcggacgctgggaccggcgagaccgcgcgagcagcgggcgggagtccggcagttggctgcaccactcgggggcccgtagtctcggcgcccctgggagccgcccggtggaagctcctgcggcaggttctgaagcaaaaacacctggatgattgcctgcgacatgtatctgtaagaagatttgaatcatttaatctgttttcagtaacagaaggcaaagaaagggaaactgaagaggaggttggtgcatgggtccaatatacaagcatcttctgtcctgaatacagtatctccttaaggcataatagtggatccttgaatgttgaagatgtccttaccagctttgacaatacaggaaatgtttgcatctggccatctgaagaggttttggcttactactgcctcaagcacaataatatattcagggcccttgctgtgtgtgagctagggggtggcatgacatgcttggctgggctcatggttgctatttctgcagatgtcaaagaagttctgttaactgatgggaatgaaaaggccatcagaaatgtgcaagacatcatcacaaggaatcagaaggctggtgtgtttaagacccagaaaatatcaagctgctttttacgatgggataatgagacagatgtctctcaactggaaggacattttgacattgttatgtgtgctgactgcctgtttctggaccagtacagagccagccttgttgatgcaataaagagattactccagcccagggggaaagcgatggtatttgccccacgccgagggaatactttaaaccagttttgcaatctagctgaaaaagctggtttctgtatccaaagacatgaaaattatgatgaacacatttcaaacttccactccaagttgaaaaaggaaaacccggacatatatgaagaaaaccttcattacccgcttctgcttattttgaccaaacatggatagaagattaagcttctcaaagacgaagaaatgtatcaagtgcatagggaatatttttacaaaaacggaaatctgtaaggggtataatcgcctgcctgcgccctttgcagcatttcacgtgtgggctatggactccacctgtcctcacccacgttattccccagctgccctctccagctccctccccgcctctttttacactctgcttgttgctcgtcctgccctaaacctttgtttgtctttaaatgtgtataagctgcctgtctgtgacttgaatttgactggtgaacaaactaaatatttttccctgtaattgagacagaatttcttttgatgatacccatccctccttcatttttttttttttttggtctttgttctgttttggtggtggtagtttttaatcagtaaacccagcaaatatcatgattctttcctggttagaaaaataaataaagtgtatctttttaaaaaaaaaaaaaaaaaaaaaaaaaaaaaaaaaaaaaaaaaaaaaaaaaaaaaaaaaaaaaaa

SEQ ID NO: 13 - protein, 44 aa, Artificial Sequence
  Description of Artificial Sequence Synthetic polypeptide
ELGTVMRSLGQNPTEAELQDMINEVDADGNGTIDFPEFLTMMAR

SEQ ID NO: 14 - protein, 38 aa, Artificial Sequence
  Description of Artificial Sequence Synthetic polypeptide
SLGQNPTEAELQDMINEVDADGNGTIDFPEFLTMMARK

SEQ ID NO: 15 - protein, 37 aa, Artificial Sequence
  Description of Artificial Sequence Synthetic polypeptide
SLGQNPTEAELQDMINEVDADGNGTIDFPEFLTMMAR

SEQ ID NO: 16 - protein, 33 aa, Artificial Sequence
  Description of Artificial Sequence Synthetic polypeptide
LTDEEVDEMIREADIDGDGQVNYEEFVQMMTAK

SEQ ID NO: 17 - protein, 22 aa, Artificial Sequence
  Description of Artificial Sequence Synthetic peptide
MADQLTEEQIAEFKEAFSLFDK

SEQ ID NO: 18 - protein, 22 aa, Artificial Sequence
  Description of Artificial Sequence Synthetic peptide
EADIDGDGQVNYEEFVQMMTAK

SEQ ID NO: 19 - protein, 20 aa, Artificial Sequence
  Description of Artificial Sequence Synthetic peptide
HVMTNLGEKLTDEEVDEMIR

SEQ ID NO: 20 - protein, 21 aa, Artificial Sequence
  Description of Artificial Sequence Synthetic peptide
DGNGYISAAELRHVMTNLGEK

SEQ ID NO: 21 - protein, 17 aa, Artificial Sequence
  Description of Artificial Sequence Synthetic peptide
EAFSLFDKDGDGTITTK

SEQ ID NO: 22 - protein, 16 aa, Artificial Sequence
  Description of Artificial Sequence Synthetic peptide
VFDKDGNGYISAAELR

SEQ ID NO: 23 - protein, 16 aa, Artificial Sequence
  Description of Artificial Sequence Synthetic peptide
DGDGTITTKELGTVMR

SEQ ID NO: 24 - protein, 14 aa, Artificial Sequence
  Description of Artificial Sequence Synthetic peptide
MADQLTEEQIAEFK

SEQ ID NO: 25 - protein, 13 aa, Artificial Sequence
  Description of Artificial Sequence Synthetic peptide
DTDSEEEIREAFR

SEQ ID NO: 26 - protein, 11 aa, Artificial Sequence
  Description of Artificial Sequence Synthetic peptide
MKDTDSEEEIR

SEQ ID NO: 27 - protein, 11 aa, Artificial Sequence
  Description of Artificial Sequence Synthetic peptide
LTDEEVDEMIR

SEQ ID NO: 28 - protein, 12 aa, Artificial Sequence
  Description of Artificial Sequence Synthetic peptide
DGNGYISAAELR

SEQ ID NO: 29 - protein, 9 aa, Artificial Sequence
  Description of Artificial Sequence Synthetic peptide
DTDSEEEIR

SEQ ID NO: 30 - protein, 9 aa, Artificial Sequence
  Description of Artificial Sequence Synthetic peptide
HVMTNLGEK

SEQ ID NO: 31 - protein, 8 aa, Artificial Sequence
  Description of Artificial Sequence Synthetic peptide
EAFRVFDK

SEQ ID NO: 32 - protein, 8 aa, Artificial Sequence
  Description of Artificial Sequence Synthetic peptide
EAFSLFDK

SEQ ID NO: 33 - protein, 9 aa, Artificial Sequence
  Description of Artificial Sequence Synthetic peptide
DGDGTITTK

SEQ ID NO: 34 - protein, 7 aa, Artificial Sequence
  Description of Artificial Sequence Synthetic peptide
ELGTVMR

SEQ ID NO: 35 - protein, 4 aa, Artificial Sequence
  Description of Artificial Sequence Synthetic peptide
EAFR

SEQ ID NO: 36 - protein, 4 aa, Artificial Sequence
  Description of Artificial Sequence Synthetic peptide
VFDK

SEQ ID NO: 37 - protein, 149 aa, Artificial Sequence [sequence omitted]

SEQ ID NO: 38 - protein, 323 aa, Homo sapiens
MESRVADAGTGETARAAGGSPAVGCTTRGPVVSAPLGAARWKLLRQVLKQKHLDDCLRHVSVRRFESFNLFSVTEGKERETEEEVGAWVQYTSIFCPEYSISLRHNSGSLNVEDVLTSFDNTGNVCIWPSEEVLAYYCLKHNNIFRALAVCELGGGMTCLAGLMVAISADVKEVLLTDGNEKAIRNVQDIITRNQKAGVFKTQKISSCFLRWDNETDVSQLEGHFDIVMCADCLFLDQYRASLVDAIKRLLQPRGKAMVFAPRRGNTLNQFCNLAEKAGFCIQRHENYDEHISNFHSKLKKENPDIYEENLHYPLLLILTKHG

SEQ ID NO: 39 - protein, 323 aa, Rattus norvegicus
MESQVAVAGDGETEAEAGKGPMIDSASQGSLVSAPKGAVRWKLLRQVLKQKQLDDGLRHVSVRRFESFNLFSVTEAKNRGTEKEAGAWVQYTSIFYPEYSISLRRNSGSLSVEDVLTSFDNTGNVCIWPSEEVLAYYCLKHSHLFRDLAVCELGGGMTCLAGLMVAISADVKEVLLTDGNEKAIRNVNSIIASNKKTGVFKTQKISSCVLRWDNKTDVSQLEGHFDIVMCADCLFLDQYRASLVDAIKRLLQPSGKALVFAPRRGNTFNQFCNLAEKAGLSLQRHENYDERISNFHSKLKKEGSDVYEENLHYPLLLILTKTG

SEQ ID NO: 40 - protein, 323 aa, Bos taurus
MESQVSDAAVGQAEQTAGEGQANSGAAPGPAASASLGAARWKLLRQVLKQKHLDDCLRHISVRRFESFNLFSVTEVKKRETEEEAGAWVQYTSIFYPEYSIFVRHNSGSLNVEDVLTSFDNTGNVCIWPAEEVLAYYCLKHSGIFRDLAVCELGGGMTCLAGLMVAISADVKEVLLTDGNEKAIRNVRDIIARNQKAGVFKTGNISSCVLRWDNETDVSQLEGHFDIVMCADCLFLDQYRASLVDAIKRLLQPRGKAMVFAPRRGNTLNQFCNLAEKAGFSIQRHENYDEHISNFHSKLKKENQDVYEENLHFPLLLILTKDG

SEQ ID NO: 41 - protein, 318 aa, Xenopus laevis
MESSVPLGNSVRTGAACGRIMAVPSKAAQGDARARWKLLGQALRKERLDDSLQKVSVRRFNSFRLFSVVEMKEIKREADCQTWFQYTCVFCPQYSLCLRHNSGISNVADILTSFDNTGNVCVWPSEEVMAYYCLKHKDIFRGLAVCELGGGMTCLAGLMVAISADVKEVLLTDGNEKAIKNVSDIIRRPQNEEMFKDRLVSSRVLRWDNETDVSQLEGHFDIVICADCLFLDQYRACLVDAIKRLLKPSGKAMVFAPPRGNTLSQFCNLAEAAGFSIQRHENYDEHISNFHSKLKEKEAAVYDENLHYPFLLVLCKTR

SEQ ID NO: 42 - protein, 308 aa, Gallus gallus
MAETAGSPRRSTADRSGGSGAGAARARWRLLGQVLKKKHLKDVHLQQVSVRRFTSFNLFSVDGQDTEEETGIWVQYKSIFYPEYSVSIRLHNGLLNVKDVLTSFDNTGNVCLWPSEEVLAYYCLKHREIFRDLAVCELGGGMTCLAGLMVAISADVKEVLLTDGNEKAIKNVSDIITRNVIAGVFKTQKVSSCILRWDNETDVSQLEGHFDIVMCADCLFLDRYRASLVDAIKRLLQPSGKAMVFAPHRGNTLNQFCNLAEKAGFSIQRHENYDEHISNFHSKLKNEEKDTYDENLHYPLLLVLTKHG

SEQ ID NO: 43 - protein, 304 aa, Arabidopsis thaliana
MDPTSSSSSALRWKILRQALLRRSDSQSQTETKRISRKATQGFNLIPCQVVDSSPQSDKSREASVCYTLPITGSPKLYLTQRVDNCSDLNDFEISNRYNIDNTGLVCQWPSEEVLAYFCKSQPERFRGKRVIELGSGYGLAGLVIAAATEASEVVISDGNPQVVNYIKRNIETNSMAFGGTSVKAMELHWNQHQLSELTNTFDIIVASDCTFFKEFHKDLARTIKMLLKAKKASEALFFSPKRGDSLEKFMKEIKDIGLHYILTENYDAQVWKRHETLVKGDEAWPNYDKNHCYPLLIQITNQI

SEQ ID NO: 44 - protein, 290 aa, Tribolium castaneum
MDQKDLVVVKNEAKKVARRRWAILAKALKSPVGSEPSSPTDEFSLRRISSFMLLQTQQLRPHDHKRTWYSYSIRIGLSEYSIVIGHRIRTFSAEDLMGFNNTGNICIWPSEETLSYYVCSNLAQFADKTILELGGGMSCLAGLFAAKYAAPKAVTVTDGNKHSVENVQAALDYNQFACPVDCKLLKWGSHEGPLYDVILCADCLFFDDARADLIECLWGCLDARGVAFVMAPKRGGTLDHFIAQSEIKGFKCRKIVNYNQVVWEKRLALIEHCEYDDDIHYPILIEVTKV

SEQ ID NO: 45 - protein, 4 aa, Unknown
  Description of Unknown Calmodulin protein motif
GGGM

SEQ ID NO: 46 - protein, 4 aa, Unknown
  Description of Unknown Calmodulin protein motif
DCLF

SEQ ID NO: 47 - protein, 323 aa, Artificial Sequence [sequence omitted]

SEQ ID NO: 48 - protein, 323 aa, Homo sapiens; identical to SEQ ID NO: 38